(19) United States
(12) Patent Application Publication
(10) Pub. No.: US 2009/0041713 A1
(43) Pub. Date: Feb. 12, 2009

(54) Hair Care Composition

(75) Inventor: Cheryl Anne Taylor, Bebington (GB)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/221,852

(22) Filed: Aug. 7, 2008

(30) Foreign Application Priority Data
Aug. 10, 2007 (EP) EP07114203

(51) Int. Cl. A61K 8/92; A61Q 5/00

(52) U.S. Cl. 424/70.27; 424/70.1

(57) ABSTRACT

A method for relaxing hair comprising the following steps:
i) applying to the hair for a sufficient period of time to lanthionize the hair a relaxer composition;
ii) terminating the lanthionization process;
iii) applying to the lanthionized hair a post-lanthionization composition comprising a phospholipid.

The present invention is directed to a hair relaxing composition and a method of relaxing hair.

Hair relaxers are compositions used to relax or straighten curly or kinky hair. Most hair relaxers straighten hair by disrupting disulfide bonds of the hair fibres with an alkaline agent or reducing agent. The chemical disruption of disulfide bonds is usually combined with mechanical straightening of the hair (e.g. by combing). The straightening process is generally terminated by rinsing and/or the application of a neutralizing composition.

A source of hydroxide ions is usually the preferred alkaline agent used to straighten hair. The term “lanthionizing” is used when referring to hair relaxed or straightened by hydroxide ions, as the straightening reaction sequence with hydroxide ions results in lanthionine residue formation.

Most frequently, commercial relaxing compositions are in the form of gels or emulsions and contain varying proportions of strong water-soluble bases, such as sodium hydroxide (NaOH). Also used are slightly-soluble metal hydroxides, such as calcium hydroxide (Ca(OH)₂), which can be converted in situ to soluble bases, such as guanidine hydroxide.

A key problem with hair relaxers is that they leave the hair treated therewith feeling rough, unconditioned and brittle. The present invention relates to hair relaxing systems which leave the hair less prone to breakage and feeling smooth.

In one aspect the present invention relates to a method for relaxing hair comprising the following steps:
i) applying to the hair for a sufficient period of time to lanthionize the hair a relaxer composition;
ii) terminating the lanthionization process;
iii) applying to the lanthionized hair a post-lanthionization composition comprising a phospholipid.

A further aspect of the invention is the use of a phospholipid as a post-treatment composition for decreasing hair breakage.

As disclosed above the present invention relates to post-treatment composition for application after application of a relaxing composition. In the context of the present invention “relaxing composition” means a composition comprising at least one hydroxide ion generator in an amount sufficient to effect lanthionization of keratin fibres. The term post-treatment refers to a treatment applied to the hair after the lathionization process, and preferably after any subsequent termination process.

The hydroxide ion generator may be chosen from those compositions that produce hydroxide ions appropriate for the lanthionization of hair. As used herein, “hydroxide ion generator” refers to both compounds and compositions that generate hydroxide ions, and compounds and compositions that comprise hydroxide ions. Hydroxide ion generators may, for example, be chosen from traditional “lye” and “no lye” hair relaxer compositions and other soluble or slightly soluble hydroxide ion sources. Preferably the hydroxide ions are generated in situ. Preferred hydroxide ion generators are strong water-soluble bases, particularly preferred is sodium hydroxide.

The post lanthionization composition comprises a phospholipid. The phospholipids are preferably complex lipids in which one of the primary hydroxyl groups of glycerin is esterified with phosphoric acid which carries an additional ester grouping. The two remaining hydroxyl groups are esterified with long chain, saturated or unsaturated fatty acids.

A suitable phospholipid is Ceramax (trademark ex Quest).

The level of phospholipid within the post-treatment composition is preferably from 1 to 10 wt %, more preferably from 3.5 to 7.5 wt % of the total composition.

The post treatment composition will preferably comprise one or more conditioning surfactants which are cosmetically acceptable and suitable for topical application to the hair.

Suitable conditioning surfactants are selected from cationic surfactants, used singly or in admixture.

Cationic surfactants useful in compositions of the invention contain amino or quaternary ammonium hydrophilic moieties which are positively charged when dissolved in the aqueous composition of the present invention.

Examples of suitable cationic surfactants are those corresponding to the general formula:

[N(R₁)(R₂)(R₃)(R₄)]⁺(X)⁻

in which R₁, R₂, R₃, and R₄ are independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

The aliphatic groups can contain, in addition to carbon and hydrogen atoms, ether linkages, and other groups such as amino groups. The longer chain aliphatic groups, e.g., those of about 12 carbons, or higher, can be saturated or unsaturated.

The most preferred cationic surfactants for conditioner compositions of the present invention are monoalkyl quaternary ammonium compounds in which the alkyl chain length is C8 to C14.

Suitable examples of such materials correspond to the general formula:

[N(R₅)(R₆)(R₇)(R₈)]⁺(X)⁻

in which R₅ is a hydrocarbyl chain having 8 to 14 carbon atoms or a functionalised hydrocarbyl chain with 8 to 14 carbon atoms and containing ether, ester, amido or amino moieties present as substituents or as linkages in the radical chain, and R₆, R₇ and R₈ are independently selected from (a) hydrocarbyl chains of from 1 to about 4 carbon atoms, or (b) functionalised hydrocarbyl chains having from 1 to about 4 carbon atoms and containing one or more aromatic, ether, ester, amido or amino moieties present as substituents or as linkages in the radical chain, and X is a salt-forming anion such as those selected from halogen, (e.g. chloride, bromide), acetate, citrate, lactate, glycolate, phosphate nitrate, sulphate, and alkylsulphate radicals.

The functionalised hydrocarbyl chains (b) may suitably contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof.

Preferably the hydrocarbyl chains R₁ have 12 to 14 carbon atoms, most preferably 12 carbon atoms. They may be derived from source oils which contain substantial amounts of fatty acids having the desired hydrocarbyl chain length. For example, the fatty acids from palm kernel oil or coconut oil can be used as a source of C8 to C12 hydrocarbyl chains.

Typical monoalkyl quaternary ammonium compounds of the above general formula for use in shampoo compositions of the invention include:
(i) lauryl trimethylammonium chloride (available commercially as Arquad C35 ex-Akzo); cocodimethyl benzyl ammonium chloride (available commercially as Arquad DMCB-80 ex-Akzo)
(ii) compounds of the general formula:

[N(R₁)(R₂)((CH₂CH₂O)ₓH)((CH₂CH₂O)_{y}H)]⁺(X)⁻

in which:
x+y is an integer from 2 to 20;

R₁ is a hydrocarbyl chain having 8 to 14, preferably 12 to 14, most preferably 12 carbon atoms or a functionalised hydrocarbyl chain with 8 to 14, preferably 12 to 14, most preferably 12 carbon atoms and containing ether, ester, amido or amino moieties present as substituents or as linkages in the radical chain;

R₂ is a C₁-C₃ alkyl group or benzyl group, preferably methyl, and

X is a salt-forming anion such as those selected from halogen, (e.g. chloride, bromide), acetate, citrate, lactate, glycolate, phosphate nitrate, sulphate, methosulphate and alkylsulphate radicals.

Suitable examples are PEG-n lauryl ammonium chlorides (where n is the PEG chain length), such as PEG-2 cocomonium chloride (available commercially as Ethoquad C12 ex-Akzo Nobel); PEG-2 cocobenzyl ammonium chloride (available commercially as Ethoquad CB/12 ex-Akzo Nobel); PEG-5 cocomonium methosulphate (available commercially as Rewoquat CPEM ex-Rewo); PEG-15 cocomonium chloride (available commercially as Ethoquad C/25 ex-Akzo).
(iii) compounds of the general formula:

[N(R₁)(R₂)(R₃)((CH₂)ₙOH)]⁺(X)⁻

in which:

n is an integer from 1 to 4, preferably 2;

R₁ is a hydrocarbyl chain having 8 to 14, preferably 12 to 14, most preferably 12 carbon atoms;

R₂ and R₃ are independently selected from C₁-C₃ alkyl groups, and are preferably methyl, and

X is a salt-forming anion such as those selected from halogen, (e.g. chloride, bromide), acetate, citrate, lactate, glycolate, phosphate nitrate, sulphate, and alkylsulphate radicals.

Suitable examples are lauryldimethylhydroxyethylammonium chloride (available commercially as Prapagen HY ex-Clariant).

Mixtures of any of the foregoing cationic surfactants compounds may also be suitable.

Examples of suitable cationic surfactants include: quaternary ammonium chlorides, e.g. alkyltrimethylammonium chlorides wherein the alkyl group has from about 8 to 22 carbon atoms, for example octyltrimethylammonium chloride, dodecyltrimethylammonium chloride, hexadecyltrimethyl-ammonium chloride, cetyltrimethylammonium chloride, octyldimethylbenzylammonium chloride, decyldimethylbenzyl-ammonium chloride, stearyldi-methylbenzylammonium chloride, didodecyldimethylammonium chloride, dioctadecyldimethylammonium chloride, tallow trimethylammonium chloride, cocotrimethylammonium chloride, and the corresponding salts thereof, e.g., bromides, hydroxides. Cetylpyridinium chloride or salts thereof, e.g., chloride

### Quaternium −5

### Quaternium −31

### Quaternium −18

and mixtures thereof.

In the conditioners of the invention, the level of cationic surfactant is preferably from 0.01 to 10, more preferably 0.05 to 7, most preferably 0.5 to 5 wt % of the total composition.

The weight ratio of cationic conditioning agent to monoglyceride within the post-treatment composition is preferably from 1:10 to 10:1, more preferably from 1:5 to 5:1.

Post treatment compositions of the invention preferably additionally comprise a fatty alcohol material. The combined use of fatty alcohol materials and cationic surfactants in conditioning compositions is believed to be especially advantageous, because this leads to the formation of a lamellar phase, in which the cationic surfactant is dispersed.

By “fatty alcohol material” is meant a fatty alcohol, an alkoxylated fatty alcohol, or a mixture thereof.

Representative fatty alcohols comprise from 8 to 22 carbon atoms, more preferably 16 to 20. Examples of suitable fatty alcohols include cetyl alcohol, stearyl alcohol and mixtures thereof. The use of these materials is also advantageous in that they contribute to the overall conditioning properties of compositions of the invention.

Alkoxylated, (e.g. ethoxylated or propoxylated) fatty alcohols having from about 12 to about 18 carbon atoms in the alkyl chain can be used in place of, or in addition to, the fatty alcohols themselves. Suitable examples include ethylene glycol cetyl ether, polyoxyethylene (2) stearyl ether, polyoxyethylene (4) cetyl ether, and mixtures thereof.

The level of fatty alcohol material in conditioners of the invention is suitably from 0.01 to 15, preferably from 0.1 to 10, and more preferably from 0.1 to 5 wt %. The weight ratio of cationic surfactant to fatty alcohol is suitably from 10:1 to 1:10, preferably from 4:1 to 1:8, optimally from 1:1 to 1:7, for example 1:3.

Post treatment compositions of the invention can also contain a cationic polymer.

The compositions of the invention can contain, emulsified droplets of a silicone conditioning agent, for enhancing conditioning performance. The silicone is insoluble in the aqueous matrix of the composition and so is present in an emulsified form, with the silicone present as dispersed droplets.

Suitable silicones include polydiorganosiloxanes, in particular polydimethylsiloxanes which have the CTFA designation dimethicone. Also suitable for use compositions of the invention (particularly shampoos and conditioners) are polydimethyl siloxanes having hydroxyl end groups, which have the CTFA designation dimethiconol. Also suitable for use in compositions of the invention are silicone gums having a slight degree of cross-linking, as are described for example in WO 96/31188. These materials can impart body, volume and stylability to hair, as well as good wet and dry conditioning.

The viscosity of the emulsified silicone itself (not the emulsion or the final hair conditioning composition) is typically at least 10,000 cst. In general we have found that conditioning performance increases with increased viscosity. Accordingly, the viscosity of the silicone itself is preferably at least 60,000 cst, most preferably at least 500,000 cst, ideally at least 1,000,000 cst. Preferably the viscosity does not exceed 10⁹ cst for ease of formulation.

Emulsified silicones for use in the shampoo compositions of the invention will typically have an average silicone droplet size in the composition of less than 30, preferably less than 20, more preferably less than 10 μm. We have found that reducing the droplet size generally improves conditioning performance. Most preferably the average silicone droplet size of the emulsified silicone in the composition is less than 2 μm, ideally it ranges from 0.01 to 1 μm. Silicone emulsions having an average silicone droplet size of ≦0.15 μm are generally termed microemulsions.

Suitable silicone emulsions for use in the invention are also commercially available in a pre-emulsified form.

Examples of suitable pre-formed emulsions include emulsions DC2-1766, DC2-1784, and microemulsions DC2-1865 and DC2-1870, all available from Dow Corning. These are all emulsions/microemulsions of dimethiconol. Cross-linked silicone gums are also available in a pre-emulsified form, which is advantageous for ease of formulation. A preferred example is the material available from Dow Corning as DC X2-1787, which is an emulsion of cross-linked dimethiconol gum. A further preferred example is the material available from Dow Corning as DC X2-1391, which is a microemulsion of cross-linked dimethiconol gum.

A further preferred class of silicones for inclusion in shampoos and conditioners of the invention are amino functional silicones. By “amino functional silicone”, is meant a silicone containing at least one primary, secondary or tertiary amine group, or a quaternary ammonium group.

Examples of suitable amino functional silicones include
(i) polysiloxanes having the CTFA designation “amodimethicone”, and the general formula:

HO—[Si(CH₃)₂—O—]ₓ—[Si(OH)(CH₂CH₂CH₂—NH—CH₂CH₂NH₂)—O—]_{y}—H

in which x and y are numbers depending on the molecular weight of the polymer, generally such that the molecular weight is between about 5,000 and 500,000.
(ii) polysiloxanes having the general formula:

R′ₐG₃₋ₐ-Si(OSiG₂)ₙ(OSiG_{b}R′_{2-b})ₘ—O—SiG₃₋ₐ-R′ₐ

in which:
G is selected from H, phenyl, OH or C₁₋₈ alkyl, e.g. methyl;
a is 0 or an integer from 1 to 3, preferably 0;
b is 0 or 1, preferably 1;
m and n are numbers such that (m+n) can range from 1 to 2000, preferably from 50 to 150;
m is a number from 1 to 2000, preferably from 1 to 10;
n is a number from 0 to 1999, preferably from 49 to 149, and

R′ is a monovalent radical of formula —C_{q}H_{2q}L in which q is a number from 2 to 8 and L is an aminofuctional group selected from the following:

—NR″—CH₂—CH₂—N(R″)₂

—N(R″)₂

—N⁺(R″)₃A⁻

—N⁺H(R″)₂A⁻

—N⁺H₂(R″)A⁻

—N(R″)—CH₂—CH₂—N⁺H₂(R″)A⁻

in which R″ is selected from H, phenyl, benzyl, or a saturated monovalent hydrocarbon radical, e.g. C₁₋₂₀ alkyl, and A is a halide ion, e.g. chloride or bromide.

Suitable amino functional silicones corresponding to the above formula include those polysiloxanes termed “trimethylsilylamodimethicone” as depicted below, and which are sufficiently water insoluble so as to be useful in compositions of the invention:

Si(CH3)3-O—[Si(CH3)2-O-]x-[Si(CH3)(R—NH—CH2CH2NH2)-O-]y-Si(CH3)3

wherein x+y is a number from about 50 to about 500, and wherein R is an alkylene group having from 2 to 5 carbon atoms. Preferably, the number x+y is in the range of from about 100 to about 300.
(iii) quaternary silicone polymers having the general formula:

{(R¹)(R²)(R³)N⁺CH₂CH(OH)CH₂O(CH₂)₃[Si(R⁴)(R⁵)—O—]ₙ—Si(R⁶)(R⁷)—(CH₂)₃—O—CH₂CH(OH)CH₂N⁺(R⁸)(R⁹)(R¹⁰)}(X⁻)₂

wherein R¹ and R¹⁰ may be the same or different and may be independently selected from H, saturated or unsaturated long or short chain alk(en)yl, branched chain alk(en)yl and C₅-C₈ cyclic ring systems;

R² thru'R⁹ may be the same or different and may be independently selected from H, straight or branched chain lower alk(en)yl, and C₅-C₈ cyclic ring systems;

n is a number within the range of about 60 to about 120, preferably about 80, and

X⁻ is preferably acetate, but may instead be for example halide, organic carboxylate, organic sulphonate or the like. Suitable quaternary silicone polymers of this class are described in EP-A-0 530 974.

Amino functional silicones suitable for use in shampoos and conditioners of the invention will typically have a mole % amine functionality in the range of from about 0.1 to about 8.0 mole %, preferably from about 0.1 to about 5.0 mole %, most preferably from about 0.1 to about 2.0 mole %. In general the amine concentration should not exceed about 8.0 mole % since we have found that too high an amine concentration can be detrimental to total silicone deposition and therefore conditioning performance.

The viscosity of the amino functional silicone is not particularly critical and can suitably range from about 100 to about 500,000 cst.

Specific examples of amino functional silicones suitable for use in the invention are the aminosilicone oils DC2-8220, DC2-8166, DC2-8466, and DC2-8950-114 (all ex Dow Corning), and GE 1149-75, (ex General Electric Silicones).

Also suitable are emulsions of amino functional silicone oils with non ionic and/or cationic surfactant.

Suitably such pre-formed emulsions will have an average amino functional silicone droplet size in the shampoo composition of less than 30, preferably less than 20, more preferably less than 10 μm. Again, we have found that reducing the droplet size generally improves conditioning performance. Most preferably the average amino functional silicone droplet size in the composition is less than 2 μm ideally it ranges from 0.01 to 1 μm.

Pre-formed emulsions of amino functional silicone are also available from suppliers of silicone oils such as Dow Corning and General Electric. Specific examples include DC929 Cationic Emulsion DC939 Cationic Emulsion, and the non-ionic emulsions DC2-7224, DC2-8467, DC2-8177 and DC2-8154 (all ex Dow Corning).

An example of a quaternary silicone polymer useful in the present invention is the material K3474, ex Goldschmidt.

Compositions according to the present invention may also comprise a dispersed, non-volatile, water-insoluble oily conditioning agent.

This component will be dispersed in the composition in the form of droplets, which form a separate, discontinuous phase from the aqueous, continuous phase of the composition. In other words, the oily conditioning agent will be present in the shampoo composition in the form of an oil-in-water emulsion.

By “insoluble” is meant that the material is not soluble in water (distilled or equivalent) at a concentration of 0.1% (w/w), at 250° C.

Suitably, the D_{3,2} average droplet size of the oily conditioning component is at least 0.4, preferably at least 0.8, and more preferably at least 1 μm. Additionally, the D_{3,2} average droplet size of the oily conditioning component is preferably no greater than 10, more preferably no greater 8, more preferably no greater than 5, yet more preferably no greater than 4, and most preferably no greater than 3.5 μm.

The oily conditioning agent may suitably be selected from oily or fatty materials, and mixtures thereof.

Oily or fatty materials are preferred conditioning agents in the shampoo compositions of the invention for adding shine to the hair and also enhancing dry combing and dry hair feel.

Preferred oily and fatty materials will generally have a viscosity of less than 5 Pa·s, more preferably less than 1 Pa·s, and most preferably less than 0.5 Pa·s, e.g. 0.1 Pa·s and under as measured at 25° C. with a Brookfield Viscometer (e.g. Brookfield RV) using spindle 3 operating at 100 rpm.

Oily and fatty materials with higher viscosities may be used. For example, materials with viscosities as high as 65 Pa·s may be used. The viscosity of such materials (i.e. materials with viscosities of 5 Pa·s and greater) can be measured by means of a glass capillary viscometer as set out further in Dow Corning Corporate Test Method CTM004, Jul. 20, 1970.

Suitable oily or fatty materials are selected from hydrocarbon oils, fatty esters and mixtures thereof.

Hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will preferably contain from about 12 to about 30 carbon atoms. Branched chain hydrocarbon oils can and typically may contain higher numbers of carbon atoms. Also suitable are polymeric hydrocarbons of alkenyl monomers, such as C₂-C₆ alkenyl monomers. These polymers can be straight or branched chain polymers. The straight chain polymers will typically be relatively short in length, having a total number of carbon atoms as described above for straight chain hydrocarbons in general. The branched chain polymers can have substantially higher chain length. The number average molecular weight of such materials can vary widely, but will typically be up to about 2000, preferably from about 200 to about 1000, more preferably from about 300 to about 600.

Specific examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, and mixtures thereof. Branched-chain isomers of these compounds, as well as of higher chain length hydrocarbons, can also be used. Exemplary branched-chain isomers are highly branched saturated or unsaturated alkanes, such as the permethyl-substituted isomers, e.g., the permethyl-substituted isomers of hexadecane and eicosane, such as 2,2,4,4,6,6,8,8-dimethyl-10-methylnondecane and 2,2,4,4,6,6-dimethyl-8-methylnonane, sold by Permethyl Corporation. A further example of a hydrocarbon polymer is polybutene, such as the copolymer of isobutylene and butene. A commercially available material of this type is L-14 polybutene from Amoco Chemical Co. (Chicago, Ill., U.S.A.).

Particularly preferred hydrocarbon oils are the various grades of mineral oils. Mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons ranging from C₁₆H₃₄ to C₂₁H₄₄. Suitable commercially available materials of this type include Sirius M85 and Sirius M125, all available from Silkolene.

Suitable fatty esters are characterised by having at least 10 carbon atoms, and include esters with hydrocarbyl chains derived from fatty acids or alcohols, e.g., monocarboxylic acid esters, polyhydric alcohol esters, and di- and tricarboxylic acid esters. The hydrocarbyl radicals of the fatty esters hereof can also include or have covalently bonded thereto other compatible functionalities, such as amides and alkoxy moieties, such as ethoxy or ether linkages.

Monocarboxylic acid esters include esters of alcohols and/or acids of the formula R′COOR in which R′ and R independently denote alkyl or alkenyl radicals and the sum of carbon atoms in R′ and R is at least 10, preferably at least 20.

Specific examples include, for example, alkyl and alkenyl esters of fatty acids having aliphatic chains with from about 10 to about 22 carbon atoms, and alkyl and/or alkenyl fatty alcohol carboxylic acid esters having an alkyl and/or alkenyl alcohol-derived aliphatic chain with about 10 to about 22 carbon atoms, benzoate esters of fatty alcohols having from about 12 to 20 carbon atoms.

The monocarboxylic acid ester need not necessarily contain at least one chain with at least 10 carbon atoms, so long as the total number of aliphatic chain carbon atoms is at least 10. Examples include isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Di- and trialkyl and alkenyl esters of carboxylic acids can also be used. These include, for example, esters of C4-CB dicarboxylic acids such as C₁-C₂₂ esters (preferably C₁-C₆) of succinic acid, glutaric acid, adipic acid, hexanoic acid, heptanoic acid, and octanoic acid. Examples include diisopropyl adipate, diisohexyl adipate, and diisopropyl sebacate. Other specific examples include isocetyl stearoyl stearate, and tristearyl citrate.

Polyhydric alcohol esters include alkylene glycol esters, for example ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol monostearate, ethoxylated propylene glycol monostearate, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, polyoxyethylene sorbitan fatty acid esters and mono-, di- and triglycerides.

Particularly preferred fatty esters are mono-, di- and triglycerides, more specifically the mono-, di-, and tri-esters of glycerol and long chain carboxylic acids such as C₁-C₂₂ carboxylic acids. A variety of these types of materials can be obtained from vegetable and animal fats and oils, such as coconut oil, castor oil, safflower oil, sunflower oil, cottonseed oil, corn oil, olive oil, cod liver oil, almond oil, avocado oil, palm oil, sesame oil, peanut oil, lanolin and soybean oil. Synthetic oils include triolein and tristearin glyceryl diiaurate.

Specific examples of preferred materials include cocoa butter, palm stearin, sunflower oil, soyabean oil and coconut oil.

The oily or fatty material is suitably present at a level of from 0.05 to 10, preferably from 0.2 to 5, more preferably from about 0.5 to 3 wt %.

In hair treatment compositions containing a conditioning agent, it is preferred that a cationic polymer also be present.

The compositions of the present invention may also contain adjuvants suitable for hair care. Generally such ingredients are included individually at a level of up to 2, preferably up to 1 wt % of the total composition.

Among suitable hair care adjuvants, are:
(i) natural hair root nutrients, such as amino acids and sugars. Examples of suitable amino acids include arginine, cysteine, glutamine, glutamic acid, isoleucine, leucine, methionine, serine and valine, and/or precursors and derivatives thereof. The amino acids may be added singly, in mixtures, or in the form of peptides, e.g. di- and tripeptides. The amino acids may also be added in the form of a protein hydrolysate, such as a keratin or collagen hydrolysate. Suitable sugars are glucose, dextrose and fructose. These may be added singly or in the form of, e.g. fruit extracts. A particularly preferred combination of natural hair root nutrients for inclusion in compositions of the invention is isoleucine and glucose. A particularly preferred amino acid nutrient is arginine.
(ii) hair fibre benefit agents. Examples are:
   ceramides, for moisturising the fibre and maintaining cuticle integrity. Ceramides are available by extraction from natural sources, or as synthetic ceramides and pseudoceramides. A preferred ceramide is Ceramide II, ex Quest. Mixtures of ceramides may also be suitable, such as Ceramides LS, ex Laboratoires Serobiologiques.

This post treatment composition may be in any form preferably in the form chosen from emulsions, solutions, suspensions, gels, creams, and pastes.

The compositions of the present invention may be provided as a multicomponent kit for straightening hair comprising at least two separate components. A first component of the kit contains comprises at least one composition for generating hydroxide ions to relax the hair. The second component comprises at least one composition comprising at least one basic amino acid.

A preferred method of terminating the lanthionization process is by rinsing the hair with water.

The invention will now be illustrated by the following non-limiting Examples.

### EXAMPLES

TABLE 1

| | | | Wt % | Wt % |
|---|---|---|---|---|
| Trade Name | Chemical Name | Supplier | Example A | Example 1 |
| Laurex CS | Cetyl/stearyl Alcohol | Huntsman | 4.80 | 4.80 |
| Perfecta | Petroleum Jelly - White | Crompton | 0.10 | 0.10 |
| Petrolatum | | | | |
| Superla No 7 | White Mineral Oil | Amoco | 0.25 | 0.25 |
| CrodalanLA | Cetyl Acetate and | Croda | 0.90 | 0.90 |
| | Stearyl Acetate and | | | |
| | Oleyl Acetate and | | | |
| | Acetylated Lanolin | | | |
| | Alcohol | | | |
| Arquad 16-29 | Cetrimonium Chloride | Akzo Nobel | 4.30 | 4.30 |
| | 29% (CTAC) | | | |
| Glycerol | Glycerol | Sigma/Aldrich | 0.50 | 0.50 |
| Natrosol | Hydroxyethylcelluslose | Hercules | 0.30 | 0.30 |
| 250HHR | | | | |
| Ceramax | Phospholipid | Quest | | 5.0 |
| Water and | | | to 100% | to 100% |
| minors | | | | |

A series of switches were treated with the compositions of table 1 immediately after the lanthionization process.

2.5 gm 6″ switches of African hair were immersed in 150 ml of 3.5% aqueous sodium hydroxide for 30 minutes at ambient conditions. The switches were then rinsed under tepid tap water for 1 minute. This process was repeated 4 times. After the final treatment the switches were placed in 150 ml of post-lanthionization composition for 15 minutes followed by rinsing and oven drying for 45 minutes at 50° C.

### Sensory Evaluation

The switches were assessed for the following sensory attributes; dryness, brittle, silkiness and gloss using the paired comparison Bradley-Terry analyses. (3 product testing).

Examples of the invention were directly compared to the comparative Examples.

| Active, Base | Non-brittle | Std Err | Non-dry | Std Err | Gloss | Std Err | Silky | Std Err |
|---|---|---|---|---|---|---|---|---|
| Example 1 | 66.45 | (18.07) | 68.10 | (18.09) | 64.55 | (19.17) | 67.85 | (18.53) |
| preference | | | | | | | | |
| scores | | | | | | | | |

Numbers refer to the percentage of panelists who selected Example 1 as scoring higher on a attribute compared with comparative Example A (control conditioner with no active).

### In-Vito Hair Breakage Test

A set of switches were treated as described above.

The weight of the switches was recorded. The switches were mounted in an automatic combing device and subjected to a total of 6 hours dry combing. The final weight was recorded.

The percentage breakage was calculated as the final weight/initial weight×100.

| Treatment | % Of Broken Fibres | Std Dev |
|---|---|---|
| Control | 24.5 | 5.2 |
| relaxed × 4 and | | |
| no post treatment | | |
| Example A Post | 5.6 | 2.2 |
| treatment | | |
| Example 1 Post | 1.0 | 1.2 |
| treatment | | |

1. A method for relaxing hair comprising the following steps:
i) applying to the hair for a sufficient period of time to lanthionize the hair a relaxer composition;
ii) terminating the lanthionization process;
iii) applying to the lanthionized hair a post-lanthionization composition comprising a phospholipid.

2. A method according to claim 1 in which the total level of phospholipid within the post-treatment composition is from 3.5 to 7.5 wt %.

3. A method according to claim 1 in which the post-treatment composition further comprises a cationic conditioning surfactant.

4. A method according to claim 3 in which the weight ratio of cationic conditioning agent to phospholipid within the post-treatment composition is from 1:5 to 5:1.

5. A method according to claim 1 in which the post-treatment composition further comprises a silicone.

6. A method according to claim 1 in which the relaxer composition comprises at least one hydroxide ion generator which generates hydroxide ions in situ.

7. Use of a phospholipid in a post-treatment composition for decreasing hair breakage.
